(19) United States
(12) Patent Application Publication
(10) Pub. No.: US 2026/0325844 A1
(43) Pub. Date: Oct. 22, 2026

(54) SYSTEM AND METHOD OF DIGITAL TWINS OF ELECTRICAL BRAIN STIMULATION

(71) Applicant: University of Florida Research Foundation, Incorporated, Gainesville, FL (US)

(72) Inventors: Ruogu FANG, Gainesville, FL (US); Skylar STOLTE, Gainesville, FL (US)

(21) Appl. No.: 19/645,995

(22) Filed: Apr. 13, 2026

Related U.S. Application Data
(60) Provisional application No. 63/789,856, filed on Apr. 16, 2025.

(51) Int. Cl. A61N 1/36; A61N 1/20

(52) U.S. Cl. CPC A61N 1/36034; A61N 1/20; A61N 1/36025

(57) ABSTRACT

Various embodiments of the present disclosure provide methods, apparatus, systems, computing devices, computing entities, and/or the like for receiving one or more neuroimaging datasets, generating one or more reference electric field maps associated with the one or more neuroimaging datasets, preprocessing one or more subject images depicting a subject brain, generating, using a regression model, one or more electric field maps corresponding to the one or more subject images, classifying, using a classification model, the one or more electric field maps into one or more electrode montages, and determining one or more electrical brain stimulation parameters corresponding to the one or more electrode montages.

### CROSS-REFERENCES TO RELATED APPLICATION(S)

The present application claims priority to U.S. Patent Application No. 63/789,856 (filed on Apr. 16, 2025), which is incorporated by reference herein in its entirety.

### GOVERNMENT LICENSE RIGHTS

This invention was made with government support under Grant No(s). 2123809 and 1908299, awarded by the National Science Foundation; and Grant No(s) R01 AG054077 and AG071469, awarded by the National Institutes of Health. The government has certain rights in the invention.

### BACKGROUND

Various embodiments of the present disclosure address technical challenges related to electrical brain stimulation techniques.

Electrical Brain Stimulation (EBS) is a non-invasive neuromodulation technique that applies low-intensity electrical currents to modulate brain activity. EBS encompasses various modalities, such as direct current, alternating current, and random noise stimulation. EBS has been used to treat clinical conditions and affiliated symptoms for decades (e.g., electroconvulsive therapy, transcranial magnetic stimulation, transcranial direct current stimulation, transcranial alternative current stimulation, transcranial random noise stimulation, deep brain stimulation, etc.). EBS techniques have demonstrated additional potential therapeutic benefits for conditions such as depression, substance use disorders, anxiety, and chronic pain. Functional digital twins in neurology are computational systems that can predict brain functional changes in response to some stimuli on an individual level.

### BRIEF SUMMARY

In general, various embodiments of the present disclosure provide methods, apparatus, systems, computing devices, computing entities, and/or the like for generating tailored electric field predictions in EBS systems.

In some embodiments, a computer-implemented method comprises: receiving one or more neuroimaging datasets; generating one or more reference electric field maps associated with the one or more neuroimaging datasets; preprocessing one or more subject images depicting a subject brain; generating, using a predictive model (e.g., regression), one or more electric field maps corresponding to the one or more subject images; classifying, using a classification model, the one or more electric field maps into one or more electrode montages; and determining one or more electrical brain stimulation parameters corresponding to the one or more electrode montages.

In some embodiments, the regression model is a SwinUNETR model architecture.

In some embodiments, the classification model is an EfficientNet classification model.

In some embodiments, the one or more reference electric field maps are generated using a Realistic vOlumetric, Approach-based Simulator for Transcranial electric stimulation (ROAST).

In some embodiments, the one or more electrical brain stimulation parameters correspond to one or more transcranial electrical stimulation operations.

In some embodiments, the one or more electrical brain stimulation parameters correspond to one or more transcranial direct current stimulation operations.

In some embodiments, the one or more subject images are MRIs.

In some embodiments, the computer-implemented method further comprises transmitting the one or more electrical brain stimulation parameters to an electro-stimulation device, wherein the electro-stimulation device is one of a transcranial magnetic stimulation device, an electroconvulsive therapy device, a deep brain stimulation device, a transcranial alternative current device, a transcranial random noise stimulation device.

In some embodiments, the one or more neuroimaging datasets are received from an imaging device, and the imaging device is one of an X-ray Computed tomography (CT) scanner, a nuclear magnetic resonance imagery (MRI) scanner or a four-dimensional computed tomography (4DCT) based imaging system.

In some embodiments, a computing apparatus comprises memory and one or more processors communicatively coupled to the memory, the one or more processors configured to perform operations comprising: receiving one or more neuroimaging datasets; generating one or more reference electric field maps associated with the one or more neuroimaging datasets; preprocessing one or more subject images depicting a subject brain; generating, using a regression model, one or more electric field maps corresponding to the one or more subject images; classifying, using a classification model, the one or more electric field maps into one or more electrode montages; and determining one or more electrical brain stimulation parameters corresponding to the one or more electrode montages.

In some embodiments, one or more non-transitory computer-readable storage media including instructions that, when executed by one or more processors, cause the one or more processors to perform operations comprising: receiving one or more neuroimaging datasets; generating one or more reference electric field maps associated with the one or more neuroimaging datasets; preprocessing one or more subject images depicting a subject brain; generating, using a regression model, one or more electric field maps corresponding to the one or more subject images; classifying, using a classification model, the one or more electric field maps into one or more electrode montages; and determining one or more electrical brain stimulation parameters corresponding to the one or more electrode montages.

### BRIEF DESCRIPTION OF THE DRAWINGS

FIG. 1 provides an example overview of a framework in accordance with some embodiments of the present disclosure.

FIG. 2 provides an example computing entity in accordance with some embodiments of the present disclosure.

FIG. 3 provides an example user computing entity in accordance with some embodiments of the present disclosure.

FIG. 4 provides an example dataflow diagram within an EBS system in accordance with some embodiments of the present disclosure.

FIG. 5A provides an example method pipeline segment in accordance with some embodiments of the present disclosure.

FIG. 5B provides an example method pipeline segment in accordance with some embodiments of the present disclosure.

FIG. 5C provides an example method pipeline segment in accordance with some embodiments of the present invention.

FIG. 6 depicts an example process for generating electrical current maps in accordance with at least some example embodiments of the present disclosure.

FIG. 7 is a flowchart diagram of an example process for determining parameter values of the electro-stimulation device on a subject to achieve improved cognitive function, in accordance with at least some example embodiments of the present invention.

### DETAILED DESCRIPTION

Various embodiments of the present disclosure are described more fully hereinafter with reference to the accompanying drawings, in which some, but not all embodiments of the present disclosure are shown. Indeed, the present disclosure may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “example” are used to be examples with no indication of quality level. Terms such as “computing,” “determining,” “generating,” and/or similar words are used herein interchangeably to refer to the creation, modification, or identification of data. Further, “based on,” “based at least in part on,” “based at least on,” “based upon,” and/or similar words are used herein interchangeably in an open-ended manner such that they do not necessarily indicate being based only on or based solely on the referenced element or elements unless so indicated. Like numbers refer to like elements throughout.

Embodiments of the present disclosure include systems, methods, computer program products, and techniques for building a nearly real-time digital twin of an individualized brain in EBS stimulation. Such digital twin generation techniques integrate several advanced components to achieve high performance with minimal hardware and enable personalized EBS operation refinement.

### I. OVERVIEW, TECHNICAL IMPROVEMENTS, AND TECHNICAL ADVANTAGES

Various embodiments of the present disclosure make important technical contributions to fields and/or environments leveraging electrical brain stimulation (EBS) techniques.

Optimizing EBS outcomes requires precise prediction of individual responses to stimulation parameters. One approach involves estimating electrical current fields in the brain, which can guide personalized intervention strategies. While physics-based simulators have contributed to this effort, they are often time-consuming, error-prone, and sensitive to issues such as poor segmentation of anatomical structures. The aforementioned challenges limit the clinical utility and scalability of physics-based simulators for estimating electrical current fields in the brain. Conventional EBS techniques apply a fixed current and electrode placement across participants, and as such, may generate inconsistent findings due to individual factors that produce a variability in response profiles. For example, a significant source of variability derives from inter-individual anatomical differences like brain atrophy, cortical folding structures, skull thickness, and brain shape. These factors may contribute to widely different response rates to the same stimulation parameters with respect to a plurality of recipients. Further, current EBS techniques may have dependencies on tools that are not open access.

To overcome these limitations, various embodiments of the present disclosure leverage digital functional twins for individualized EBS intervention. Embodiments of the present disclosure enable the direct generation of electric field maps from T1-weighted MRIs using a deep learning framework which incorporates specialized loss regularizations to enhance model generalizability and calibration across diverse anatomical scans and electrode configurations.

Various embodiments of the present disclosure allow users to input both a unique MRI and a desired electrode montage to the deep learning framework to produce tailored electric field predictions. The deep learning framework is trained according to a plurality of head models spanning a wide adult age range, thereby enabling robustness across diverse anatomical variations. Unlike traditional physics-based systems, which can require hours to compute results, the techniques as described herein deliver accurate electric field maps in minutes, thereby offering a significant improvement in efficiency and improved ability to implement determined EBS interventions with considerably less delay.

Electrical current field approximation offers a pathway for EBS (e.g., transcranial electronic stimulation (tES)) researchers to predict individual responses to specific stimulation parameters. By streamlining individualized current dose estimations, various embodiments of the present disclosure improve precision, efficacy, and accessibility of EBS interventions leveraging the disclosed techniques.

Embodiments of the present disclosure integrate digital functional twins for individualized EBS (e.g., tES) applications. Digital functional twins are computational models that predict brain function in response to stimuli, thereby generating precise electric field maps for EBS (e.g., tES) directly from T1-weighted MRIs. Some embodiments of the invention are described below in the context of electro-stimulation of the brain for purposes of improving treatment outcome (e.g. cognitive function of the brain). For purposes of this description, “electro-stimulation of the brain” or “electrical brain stimulation” (EBS) means any form of electrical brain stimulation to calculate precision dosing characteristics. This includes, but is not limited to, transcranial magnetic stimulation, electroconvulsive therapy, deep brain stimulation, transcranial direct current stimulation (tDCS), transcranial alternative current stimulation, transcranial random noise stimulation, deep brain stimulation and other forms of transcranial electrical stimulation. In addition, while the proof of concept presented specifically targeted cognitive function, this approach can also be applied in the context of any outcome measure affiliated with treatment response/outcome (e.g., cognitive, physical, mental health, neuroimaging, etc.).

Embodiments of the present invention employ specialized loss regularizations to enhance model generalizability and calibration across diverse individuals and electrode montages. The deep learning framework enables users to input unique MRIs and specify desired electrode montages, thereby enabling tailored predictions for specific stimulation configurations. By enabling fast, accurate, and user-friendly simulations, various embodiments of the present disclosure enable more effective and accessible applications of EBS (e.g., tES) in clinical and research settings.

Embodiments of the present invention improve the capacity for clinical research to model EBS parameters based on patient-wise digital twins by providing a nearly real-time digital twin of an individualized brain in EBS stimulation without relying on additional tools. The deep learning framework(s) as described herein leverage transformers to learn global spatial information. Further, the deep learning framework(s) as described herein comprise special loss regularizations to improve the framework's generalizability and calibration across individual scans and electrode montages.

Embodiments of the present disclosure enable improved speed of processing and inference relative to existing methods for generating electric field maps. By increasing the speed with which processing and inference can be completed, embodiments of the present disclosure enable real-time generation of electric field maps.

### II. COMPUTER PROGRAM PRODUCTS, METHODS, AND COMPUTING ENTITIES

Embodiments of the present disclosure may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In some embodiments, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In some embodiments, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present disclosure may also be implemented as methods, apparatus, systems, computing devices, computing entities, and/or the like. As such, embodiments of the present disclosure may take the form of an apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises combination of computer program products and hardware performing certain steps or operations.

Embodiments of the present disclosure are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some example embodiments, retrieval, loading, and/or execution may be performed in parallel such that multiple instructions are retrieved, loaded, and/or executed together. Thus, such embodiments can produce specifically configured machines performing the steps or operations specified in the block diagrams and flowchart illustrations. Accordingly, the block diagrams and flowchart illustrations support various combinations of embodiments for performing the specified instructions, operations, or steps.

### III. EXAMPLE FRAMEWORK

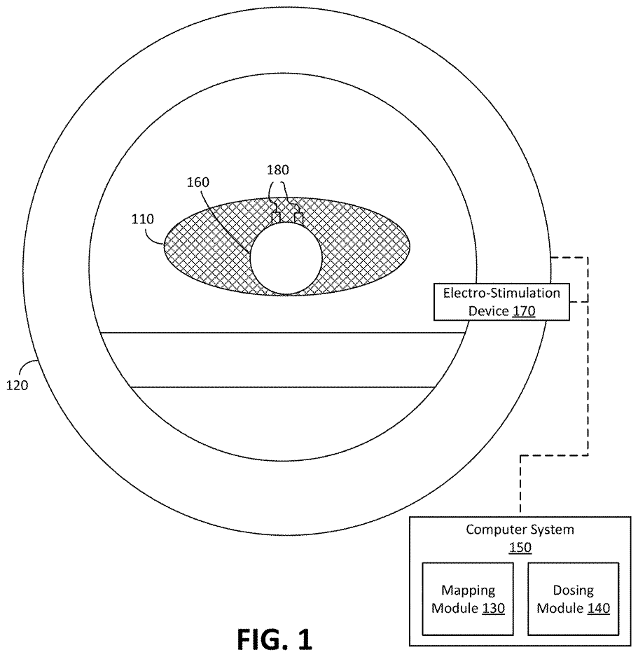

FIG. 1 provides an example overview of a framework 100 in accordance with some embodiments of the present disclosure. The framework 100 may correspond to a system for precision dosing of electrical stimulation of the brain, according to example embodiments of the present invention. For purposes of illustration, a subject 110 is depicted, but is not part of the framework 100. One or more imaging systems 120 are provided, to scan images of the subject 110 within an imaging systems volume that encompasses part of the subject 110 (e.g. at least the head 160). In an example embodiment, the volume may encompass the entire subject 110. In another example embodiment, the volume encompasses a head 160 of the subject 110. The imaging systems 120 are non-invasive and obtain cross-sectional images that are axially stacked to generate imaging data of the volume. In an example embodiment, the imaging system 120 is a first imaging device that obtains first measurements that relate to tissue type inside the volume. For example, the first imaging device is an X-ray Computed tomography CT scanner, a nuclear MRI scanner or a 4DCT, or a Single Photon Emission Computed Tomography (SPECT).

As illustrated in FIG. l, the framework 100 includes an electro-stimulation device 170 that includes a plurality of electrodes 180 positioned on a surface of the head 160 of the subject 110. In an embodiment, the electro-stimulation device 170 has one or more parameters including a location of the plurality of electrodes 180 on the head 160; a current intensity of the plurality of electrodes 180; a duration of stimulation of the head 160 with the plurality of electrodes 180; a duration of a ramp up/ramp down of the current intensity; and a number of stimulation sessions. In some embodiments, the locations of the plurality of electrodes 180 on the head 160 are based on the 10-20 system. In one embodiment, the electro-stimulation device 170 generates an electric field within the volume (e.g. within the head 160) over a plurality of volume elements or voxels that are defined within a frame of reference of the electro-stimulation device 170. In one embodiment, the electric field generated within each voxel has one or more parameter values (e.g. current intensity, direction, etc.). Although FIG. l depicts the imaging systems 120 and electro-stimulation device 170 in the framework 100, the electro-stimulation device 170 and imaging systems 120 are not necessarily in one system or apparatus and do not need to work simultaneously. Additionally, images can be captured by the imaging systems 120 before generating the electric field with the electro-stimulation device 170.

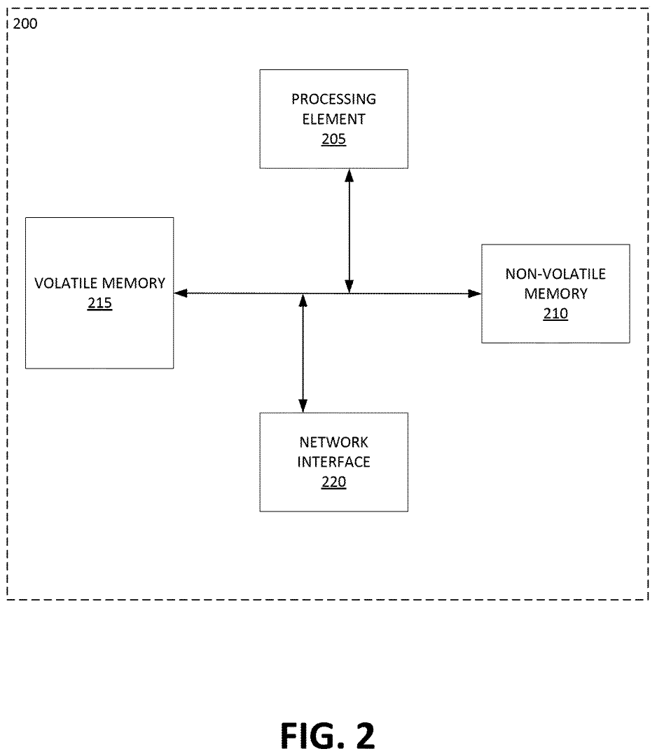

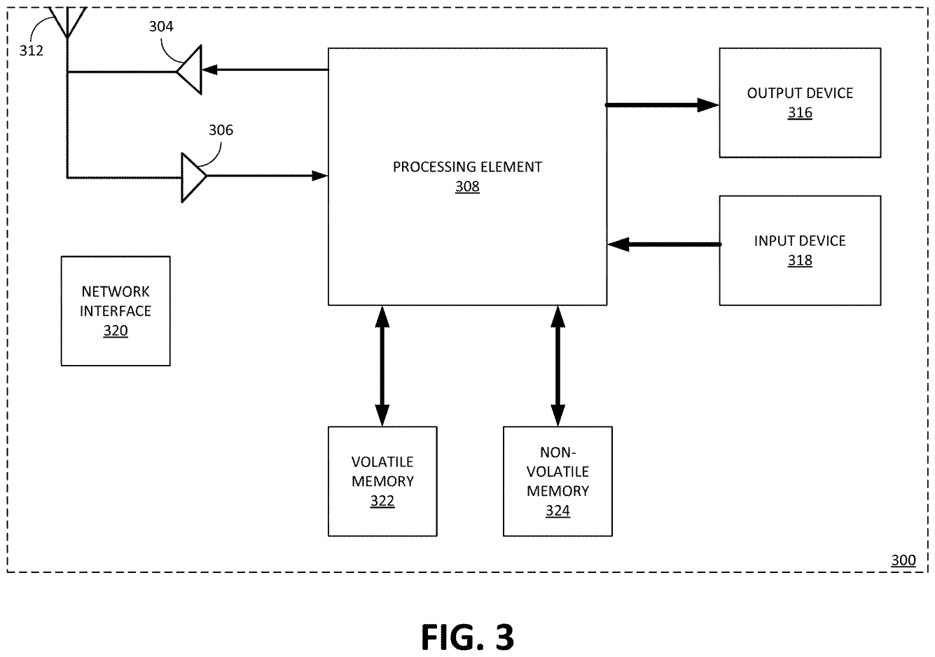

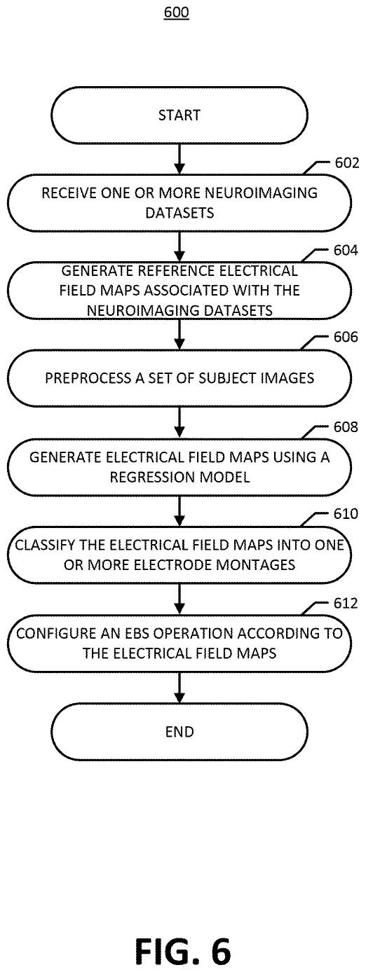

As illustrated in FIG. l, a system 150 is provided to control the one or more imaging systems 120, to collect image data from the one or more imaging systems 120 before or at the time of electrical stimulation and to control the electro-stimulation device 170. In an embodiment, the system 150 includes a dosing module 140 to perform one or more steps of a method described below with reference to FIG. 6. In another embodiment, the system 150 includes a mapping module 130 to perform one or more steps of a method described below with reference to FIG. 6. In various embodiments, the system 150 comprises one or more general purpose computer systems or upgraded computer systems and may correspond to computing entity 200 as described with respect to FIG. 2 and/or computing entity 300 as described with respect to FIG. 3.

### A. Example User Device

FIG. 2 provides an example computing entity 200 in accordance with some embodiments of the present disclosure. The computing entity 200 is an example of the system 150 of FIG. 1. In general, the terms computing entity, computer, entity, device, system, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein.

As shown in FIG. 2, in some embodiments, the computing entity 200 may include, or be in communication with, one or more processing elements 205 (also referred to as processors, processing circuitry, and/or similar terms used herein interchangeably) that communicate with other elements within the computing entity 200 via a bus, for example. As will be understood, the processing element 205 may be embodied in a number of different ways.

For example, the processing element 205 may be embodied as one or more complex programmable logic devices (CPLDs), microprocessors, multi-core processors, coprocessing entities, application-specific instruction-set processors (ASIPs), microcontrollers, and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like.

As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present disclosure when configured accordingly.

In some embodiments, the computing entity 200 may further include, or be in communication with, non-volatile media (also referred to as non-volatile storage, memory, memory storage, memory circuitry, and/or similar terms used herein interchangeably). In some embodiments, the non-volatile media may include one or more non-volatile memory 210, including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile media may store databases, database instances, database management systems, data, applications, programs, program modules, scripts, code (e.g., source code, object code, byte code, compiled code, interpreted code, machine code, etc.) that embodies one or more computer functions described herein, executable instructions, and/or the like. The term database, database instance, database management system, and/or similar terms used herein interchangeably, may refer to a collection of records or data that is stored in a computer-readable storage medium using one or more database models; such as a hierarchical database model, network model, relational model, entity-relationship model, object model, document model, semantic model, graph model, and/or the like.

In some embodiments, the computing entity 200 may further include, or be in communication with, volatile media (also referred to as volatile storage, memory, memory storage, memory circuitry, and/or similar terms used herein interchangeably). In some embodiments, the volatile media may also include one or more volatile memory 215, including, but not limited to, RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile storage or memory media may be used to store at least portions of the databases, database instances, database management systems, data, applications, programs, program modules, code (source code, object code, byte code, compiled code, interpreted code, machine code) that embodies one or more other computer functions described herein, executable instructions, and/or the like being executed by, for example, the processing element 205. Thus, the databases, database instances, database management systems, data, applications, programs, program modules, code (source code, object code, byte code, compiled code, interpreted code, machine code) that embodies one or more computer functions described herein, executable instructions, and/or the like may be used to control certain aspects of the operation of the computing entity 200 with the assistance of the processing element 205 and operating system.

As indicated, in some embodiments, the computing entity 200 may also include one or more network interfaces 220 for communicating between various computing entities such as by communicating data, code, content, information, and/or similar terms used herein interchangeably that may be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. In some embodiments, the computing entity 200 communicates with another computing entity for uploading or downloading data or code. Similarly, the computing entity 200 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the computing entity 200 may include, or be in communication with, one or more input elements, such as a keyboard input, a mouse input, a touch screen/display input, motion input, movement input, audio input, pointing device input, joystick input, keypad input, and/or the like. The computing entity 200 may also include, or be in communication with, one or more output elements (not shown), such as audio output, video output, screen/display output, motion output, movement output, and/or the like.

### B. Example External Computing Entity

FIG. 3 provides an example computing entity 300 in accordance with some embodiments of the present disclosure. In general, FIG. 3 may be representative of components of any of the modules/systems described herein generally enabling processing within each component and communication between the components. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. As shown in FIG. 3, the computing entity 300 may include an antenna 312, a transmitter 304 (e.g., radio), a receiver 306 (e.g., radio), and a processing element 308 (e.g., CPLDs, microprocessors, multi-core processors, coprocessing entities, ASIPs, microcontrollers, and/or controllers) that provides signals to and receives signals from the transmitter 304 and receiver 306, correspondingly. Generally, computing entity 300 may be an example of the system 150 of FIG. 1.

The signals provided to and received from the transmitter 304 and the receiver 306, correspondingly, may include signaling information/data in accordance with air interface standards of applicable wireless systems. In this regard, the computing entity 300 may be capable of operating with one or more air interface standards, communication protocols, modulation types, and access types. More particularly, the computing entity 300 may operate in accordance with any of a number of wireless communication standards and protocols, such as those described above with regard to the computing entity 200. In some embodiments, the computing entity 300 may operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the computing entity 300 may operate in accordance with multiple wired communication standards and protocols, such as those described above with regard to the computing entity 200 via a network interface 320.

Via these communication standards and protocols, the computing entity 300 may communicate with various other entities using mechanisms such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The computing entity 300 may also download code, changes, add-ons, and updates, for instance, to its firmware, software (e.g., including executable instructions, applications, program modules), and operating system.

The computing entity 300 may also comprise a user interface (that may include an output device 316 (e.g., display, speaker, tactile instrument, etc.) coupled to a processing element 308) and/or a user input interface (coupled to a processing element 308). For example, the user interface may be a user application, browser, user interface, and/or similar words used herein interchangeably executing on and/or accessible via the computing entity 300 to interact with and/or cause display of information/data from the computing entity 200, as described herein. The user input interface may comprise any of a plurality of input devices 318 (or interfaces) allowing the computing entity 300 to receive code and/or data, such as a keypad (hard or soft), a touch display, voice/speech or motion interfaces, or other input device. In some embodiments including a keypad, the keypad may include (or cause display of) the conventional numeric (0-9) and related keys (#, *), and other keys used for operating the computing entity 300 and may include a full set of alphabetic keys or set of keys that may be activated to provide a full set of alphanumeric keys. In addition to providing input, the user input interface may be used, for example, to activate or deactivate certain functions, such as screen savers and/or sleep modes.

The computing entity 300 may also include volatile memory 322 and/or non-volatile memory 324, which may be embedded and/or may be removable. For example, the non-volatile memory 324 may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory 322 may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile memory may store databases, database instances, database management systems, data, applications, programs, program modules, scripts, code (source code, object code, byte code, compiled code, interpreted code, machine code, etc.) that embodies one or more computer functions described herein, executable instructions, and/or the like to implement the functions of the computing entity 300. As indicated, this may include a user application that is resident on the computing entity 300 or accessible through a browser or other user interface for communicating with the computing entity 200 and/or various other computing entities.

In another embodiment, the computing entity 300 may include one or more components or functionalities that are the same or similar to those of the computing entity 200, as described in greater detail above. As will be recognized, these architectures and descriptions are provided for example purposes only and are not limited to the various embodiments.

In various embodiments, the computing entity 300 may be embodied as an artificial intelligence (AI) computing entity. Accordingly, the computing entity 300 may be configured to provide and/or receive information/data from a user via an input/output mechanism, such as a display, a camera, a speaker, a voice-activated input, and/or the like. In certain embodiments, an AI computing entity may comprise one or more predefined and executable program algorithms stored within an onboard memory storage module, and/or accessible over a network. In various embodiments, the AI computing entity may be configured to retrieve and/or execute one or more of the predefined program algorithms upon the occurrence of a predefined trigger event.

In at least some example embodiments, an appropriate hardware implementation for system 150 utilizes 1 NVIDIA A100 GPU, 4 CPUs, and 170 Gb of RAM. Such a hardware implementation may enable inference time of roughly 20 seconds per MRI. In such embodiments, the models are trained using an AdamW optimizer using a batch size of 2 for training and validation.

### C. Example EBS System

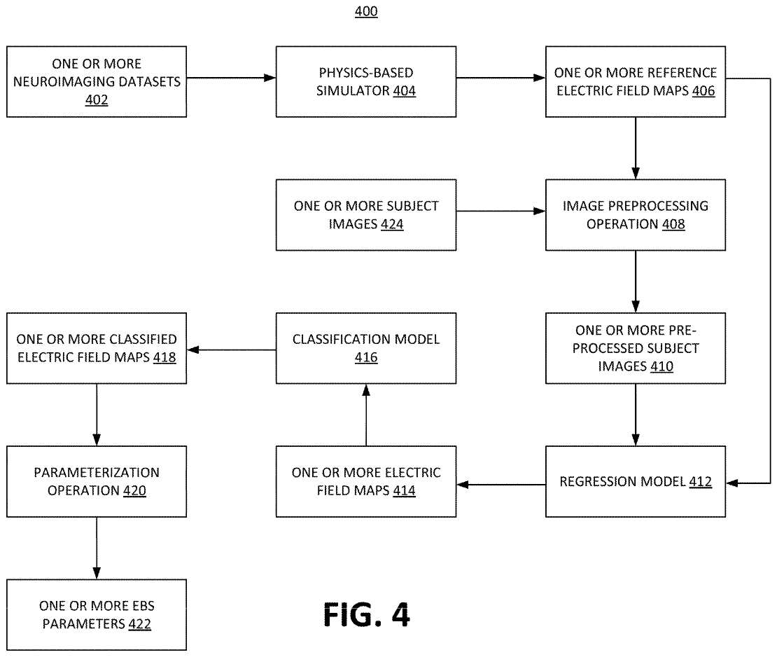

FIG. 4 provides an example dataflow diagram 400 depicting dataflow within an electrical brain stimulation (EBS) system. As depicted, the dataflow diagram 400 includes one or more neuroimaging datasets 402, a physics-based simulator 404, one or more reference electric field maps 406, an image preprocessing operation 408, one or more subject images 424, one or more pre-processed subject images 410, a regression model 412, one or more electric field maps 414, a classification model 416, one or more classified electric field maps 418, a parameterization operation 420, and one or more EBS parameters 422. The EBS system may be configured to generate optimized EBS parameters for applying EBS to a subject brain depicted by the one or more subject images 424.

The one or more neuroimaging datasets 402 may comprise three-dimensional (3D) volumetric T1-weighted magnetic resonance images (MRIs) depicting subject brains. In some embodiments, the one or more neuroimaging datasets 402 include structural T1-MRIs collected from participants across a wide adult age range, including both older adult and younger adult cohorts.

In some embodiments, the one or more neuroimaging datasets 402 are acquired using MRI scanners, such as 3-Tesla Siemens scanners with multi-channel head coils. The one or more neuroimaging datasets 402 may include images acquired using MPRAGE sequence parameters with specified repetition times, echo times, and flip angles. In some embodiments, the one or more neuroimaging datasets 402 have varying voxel sizes and fields of view depending on the acquisition protocol.

The one or more neuroimaging datasets 402 may be provided to the physics-based simulator 404 to generate the one or more reference electric field maps 406 for training purposes. In some embodiments, the one or more neuroimaging datasets 402 are also provided to the image preprocessing operation 408 to generate the one or more pre-processed subject images 410, which are then provided to the regression model 412 for generating the one or more electric field maps 414. In some embodiments, the one or more neuroimaging datasets 402 include unique individual head models that enable the system to ensure robustness across diverse anatomical variations.

The physics-based simulator 404 may apply input parameters to finite element methods to produce the one or more reference electric field maps 406. In some embodiments, the physics-based simulator 404 comprises tools such as SimNibs or ROAST, which predict the electrical current dosages that reach the target treatment areas.

The physics-based simulator 404 may receive the one or more neuroimaging datasets 402 and generate the one or more reference electric field maps 406 based on specified electrode montage configurations. In some embodiments, the physics-based simulator 404 generates reference electric field maps for common montages such as F3-F4 and C3-Fp2 based on established protocols in tDCS research.

The one or more reference electric field maps 406 may be generated by the physics-based simulator 404 using finite element methods applied to the one or more neuroimaging datasets 402. In some embodiments, the one or more reference electric field maps 406 predict the electrical current dosages that reach the target treatment areas for specified electrode montage configurations.

The one or more reference electric field maps 406 may serve as ground truth data for training the regression model 412. In some embodiments, the one or more reference electric field maps 406 are generated for common electrode montages such as F3-F4 and C3-Fp2 based on established protocols in tDCS research, with input current amplitudes of 2 mA.

The one or more reference electric field maps 406 may be compared against the one or more electric field maps 414 generated by the regression model 412 using a loss function that includes Mean Absolute Error (MAE). In some embodiments, the one or more reference electric field maps 406 enable the deep learning framework to learn to generate accurate electric field predictions that capture both the brain structure and current doses, without requiring extended runtimes of 1-3 hours per simulation associated with traditional physics-based systems.

In some embodiments, the image preprocessing operation 408 is configured to receive the one or more reference electric field maps 406 and the one or more subject images 424. The one or more subject images 424 may comprise three-dimensional (3D) volumetric T1-weighted magnetic resonance images (MRIs) depicting a subject brain prior to preprocessing. In some embodiments, the one or more subject images 424 are derived from the one or more neuroimaging datasets 402 and represent individual anatomical scans of participants across a wide adult age range.

The one or more subject images 424 may include structural T1-MRIs acquired using MRI scanners with varying acquisition parameters, voxel sizes, and fields of view. In some embodiments, the one or more subject images 424 include unique individual head models that enable the system to capture both the brain structure and current doses when generating the one or more electric field maps 414 via the regression model 412.

The image preprocessing operation 408 may receive the one or more subject images 424 and generate the one or more pre-processed subject images 410 suitable for input to the regression model 412. In some embodiments, the image preprocessing operation 408 utilizes Medical Open Network for Artificial Intelligence (MONAI) transformations so that the final model can work on inference MRIs without other software dependencies.

The image preprocessing operation 408 may normalize all MRI voxels of the one or more subject images 424 between 0-1 and crop images into 128×128×128 inputs. In some embodiments, the image preprocessing operation 408 performs augmentation on the training data derived from the one or more subject images 424, including random data flips and intensity shifts both at a probability of 0.1.

The image preprocessing operation 408 may evaluate validation and testing data from the one or more subject images 424 over the entire image inputs using 128×128×128 windows using a sliding window function. In some embodiments, the image preprocessing operation 408 transforms the one or more subject images 424 into the one or more pre-processed subject images 410, enabling the regression model 412 to generate the one or more electric field maps 414.

In some embodiments, the regression model 412 is configured to receive the one or more preprocessed subject images 410. The regression model 412 may generate the one or more electric field maps 414 through image-to-image translation. In some embodiments, the regression model 412 employs a SwinUNETR architecture that combines the advantages of U-Net with those of transformer modules in a U-Net like structure.

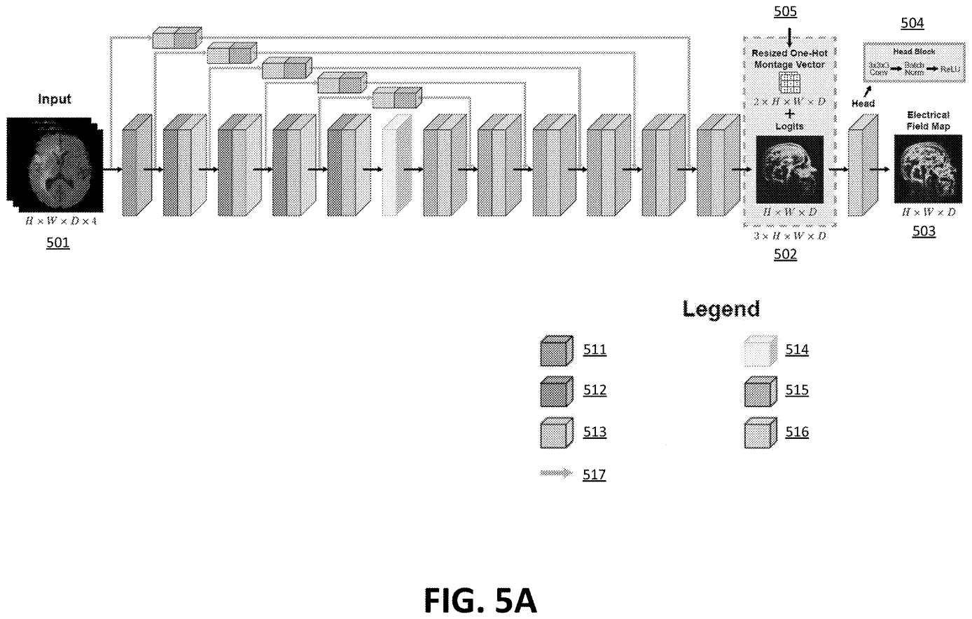

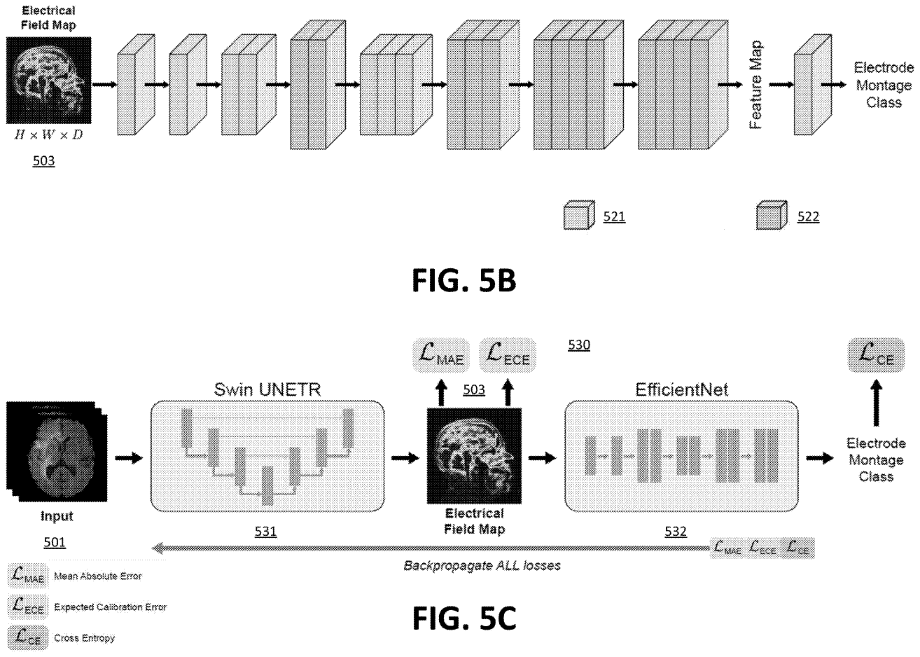

The regression model 412 may employ a transformer encoder and convolutional neural network (CNN) decoder together, where the transformer encoder includes a patch partition layer, a plurality of transformer blocks, and a plurality of patch merging layers (as described in further detail with respect to FIG. 5A). In some embodiments, the regression model 412 performs encoder feature extraction at five different resolutions using shifting windows, enabling the plurality of transformer blocks 512 to learn global spatial information through attention mechanisms.

The regression model 412 may compensate for the limited ability of convolutional neural networks (CNNs) to model global information due to the limited size of convolution kernels. In some embodiments, the decoder pathway of the regression model 412 includes a bridge layer, a plurality of residual block layers, and hidden layers (as described in further detail with respect to FIG. 5A), which progressively reconstruct the one or more electric field maps 414 from the one or more pre-processed subject images 410.

The regression model 412 may be trained using the one or more reference electric field maps 406 as ground truth data, with a loss function that includes Mean Absolute Error (MAE). In some embodiments, the regression model 412 is modified to receive a one-hot montage vector via a head block, enabling generation of electric field maps specific to a user's choice for the electrode montage.

The one or more electric field maps 414 may be generated by the regression model 412 from the one or more pre-processed subject images 410 through image-to-image translation. In some embodiments, the one or more electric field maps 414 predict the electrical current dosages that reach the target treatment areas for specified electrode montage configurations. The one or more electric field maps 414 may be three-dimensional (3D) volumetric outputs that capture both the brain structure and current doses corresponding to the one or more pre-processed subject images 410. In some embodiments, the one or more electric field maps 414 are generated in minutes, unlike traditional physics-based systems that require 1-3 hours per simulation.

The one or more electric field maps 414 may be provided to the classification model 416 to generate the one or more classified electric field maps 418. In some embodiments, the one or more electric field maps 414 are compared against the one or more reference electric field maps 406 during training using a loss function that includes Mean Absolute Error (MAE) to optimize the regression model 412.

The one or more electric field maps 414 may enable clinical research trials to determine optimal individual tDCS parameters with speed, accuracy, and reliability. In some embodiments, the one or more electric field maps 414 are tailored to specific stimulation configurations based on the one-hot montage vector 505 provided to the regression model 412 via the head block 504.

The classification model 416 may receive the one or more electric field maps 414 and generate the one or more classified electric field maps 418 by classifying the electric field maps into the different electrode montages. In some embodiments, the classification model 416 serves as an additional regularization mechanism for the regression model 412 output, classifying the output maps according to electrode montage type to help further differentiate the electric field maps that result from the same input MRI.

The classification model 416 may employ an EfficientNet framework that is designed to improve accuracy while conserving computational resources through a special model scaling scheme that balances network depth, width, and resolution. In some embodiments, the classification model 416 includes a plurality of MBConv1 blocks and a plurality of MBConv6 blocks, which employ mobile inverted bottleneck convolutional blocks that integrate squeeze-and-excitation blocks, depthwise separable convolutions, and inverted residuals.

The classification model 416 may generate logits representing the output electrode montage class predictions, which are compared against the true electrode montage class during training using cross entropy loss. In some embodiments, the entire loss function is backpropagated across both the regression model 412 and the classification model 416, enabling a dual learning scheme where the regression and classification models train simultaneously.

The one or more classified electric field maps 418 may be generated by the classification model 416 by classifying the one or more electric field maps 414 into the different electrode montages. In some embodiments, the one or more classified electric field maps 418 represent the output electrode montage class predictions associated with the one or more electric field maps 414.

The one or more classified electric field maps 418 may include logits that indicate the predicted electrode montage type, such as F3-F4 or C3-Fp2 configurations. In some embodiments, the one or more classified electric field maps 418 serve as an additional regularization for the regression model 412 output, helping to further differentiate the electric field maps that result from the same input MRI when different electrode montage configurations are applied.

The one or more classified electric field maps 418 may be compared against the true electrode montage class during training using cross entropy loss. In some embodiments, the one or more classified electric field maps 418 are provided to the parameterization operation 420 to determine the one or more EBS parameters 422 corresponding to the classified electrode montages.

The one or more classified electric field maps 418 may enable clinical research trials to determine optimal individual tDCS parameters by validating that the generated electric field maps correspond to the intended electrode montage configurations. In some embodiments, the entire loss function associated with the one or more classified electric field maps 418 is backpropagated across both the regression model 412 and the classification model 416, enabling a dual learning scheme where the regression and classification models train simultaneously.

The parameterization operation 420 may receive the one or more classified electric field maps 418 and determine the one or more EBS parameters 422 corresponding to the classified electrode montages. In some embodiments, the parameterization operation 420 enables clinical research trials to determine optimal individual tDCS parameters with speed, accuracy, and reliability. The parameterization operation 420 may iterate through possible tDCS parameters to predict positive intervention based on the one or more classified electric field maps 418. In some embodiments, the parameterization operation 420 streamlines the process of individual current dose estimations for improved tDCS interventions.

The parameterization operation 420 may determine the one or more EBS parameters 422 that account for inter-individual anatomical differences like brain atrophy, cortical folding structures, skull thickness, and brain shape. In some embodiments, the parameterization operation 420 addresses the variability in response profiles caused by individual factors, enabling customized treatment planning rather than applying fixed current and electrode placement across participants.

The parameterization operation 420 may generate the one or more EBS parameters 422 in minutes, unlike traditional physics-based systems that require 1-3 hours per simulation. In some embodiments, the parameterization operation 420 paves the path toward individualized digital functional twins for non-invasive brain stimulation by enabling users to input a desired electrode montage along with the T1-weighted MRI for a custom output.

The one or more EBS parameters 422 may be determined by the parameterization operation 420 based on the one or more classified electric field maps 418. In some embodiments, the one or more EBS parameters 422 include electrode montage configurations, such as F3-F4 or C3-Fp2 montages, and input current amplitudes for transcranial direct current stimulation (tDCS).

The one or more EBS parameters 422 may be tailored to individual anatomical variations to address inter-individual differences like brain atrophy, cortical folding structures, skull thickness, and brain shape. In some embodiments, the one or more EBS parameters 422 enable customized treatment planning rather than applying fixed current and electrode placement across participants.

The one or more EBS parameters 422 may predict the electrical current dosages that reach the target treatment areas for specified stimulation configurations. In some embodiments, the one or more EBS parameters 422 are optimized to predict positive intervention for ailments such as fibromyalgia, depression, anxiety, substance use disorder, stroke, Alzheimer's Disease, and pain.

### D. Example EBS Method Pipeline

FIG. 5A provides an example method pipeline segment 500 corresponding to a transformer encoder pathway in accordance with some embodiments of the present disclosure. As depicted, example method pipeline segment 500 includes a patch partition layer 511, a plurality of transformer blocks 512 and a plurality of patch merging layers 513, a bridge layer 514, and a plurality of residual block layers 515 and hidden layer(s) 516. Example method pipeline segment 500 shows an input MRI image 501's progression through various machine learning framework layers to ultimately provide an electric field map 503. Example method pipeline segment 500 additionally includes a plurality of indicated skip connections 517, each connecting an input item or an output item from a subject layer to a corresponding downstream hidden layer(s) 516.

Patch partition layer 511 may be responsible for dividing the input MRI image 501 into a plurality of sub-images. In at least some embodiments, patch partition layer 511 is configured to compress or flatten each of the plurality of sub-images. The patch partition layer 511 may be an initial processing stage of a regression model that receives a three-dimensional (3D) MRI input volume. In some embodiments, the patch partition layer 511 divides the input volume into a sequence of non-overlapping patches, converting the volumetric image data into a format suitable for processing by subsequent transformer blocks (e.g., the plurality of transformer blocks 512). The preprocessing may crop images into 128×128×128 inputs.

The patch partition layer 511 may provide an entry point to the transformer encoder pathway, where feature extraction may occur at multiple different resolutions using shifting windows. By partitioning the input MRI volume into discrete patches, the patch partition layer 511 enables the plurality of transformer blocks 512 to apply attention mechanisms that model global information across the spatial extent of the brain imaging data, compensating for the limited receptive field of convolution kernels in conventional convolutional neural networks. The patch partition layer 511 may operate in conjunction with the plurality of patch merging layers 513 that progressively reduce spatial resolution during encoding, and bridge components that connect the encoder pathway to the decoder pathway of the regression model.

The plurality of transformer blocks 512 may each be configured to leverage linear embedding techniques with respect to the plurality of sub-images. In at least some embodiments, each transformer block 512 consists of a multi-head self-attention (MSA) mechanism and a multilayer perceptron (MLP). Each transformer block 512 may include a normalization layer. In at least some embodiments, the plurality of transformer blocks 512 may be configured to use window-based multi-head self-attention mechanisms, such that an input feature map is divided into windows, and a subsequent window shifting operation is leveraged to enable information exchange between the windows.

In some embodiments, the plurality of transformer blocks 512 are components of the transformer encoder within a regression model that process the patches generated by the patch partition layer 511. The plurality of transformer blocks 512 may employ attention mechanisms to learn global spatial information from the input MRI image 501. Convolutional neural networks (CNNs) may lack some ability to model global information due to the limited size of convolution kernels, and the plurality of transformer blocks 512 compensate for this limitation by capturing long-range spatial dependencies across the brain imaging data.

The plurality of transformer blocks 512 may operate within an encoder pathway that performs feature extraction at five different resolutions using shifting windows. The shifting window mechanism enables the plurality of transformer blocks 512 to efficiently compute self-attention within local windows while also enabling cross-window connections, thereby balancing computational efficiency with the ability to model global context.

The plurality of transformer blocks 512 may receive the sequence of patches from the patch partition layer 511 and generate encoded feature representations for use by the plurality of patch merging layers 513. In some embodiments, the plurality of patch merging layers 513 progressively reduce spatial resolution between stages of the plurality of transformer blocks 512. The plurality of patch merging layers 513 may be components of a transformer encoder within the regression model that progressively reduce spatial resolution between successive stages of the plurality of transformer blocks 512. The plurality of patch merging layers 513 enable encoder feature extraction at five different resolutions, creating a hierarchical representation of the input MRI image 501.

Each of the plurality of patch merging layers 513 may concatenate features from groups of neighboring patches and applies a linear transformation to reduce the spatial dimensions while increasing the feature channel dimensions. This downsampling operation allows the plurality of transformer blocks 512 at subsequent stages to operate on coarser spatial resolutions with richer feature representations, enabling the capture of both fine-grained local details and broader contextual information from the brain imaging data.

The plurality of patch merging layers 513 may work in conjunction with the patch partition layer 511 and the plurality of transformer blocks 512 within the encoder pathway. The hierarchical features generated through the plurality of patch merging layers 513 are provided to the decoder pathway via the plurality of indicated skip connections 517 and the bridge layer 514, where they are progressively combined with the plurality of residual block layers 515 and the hidden layer(s) 516 to reconstruct the electric field map 503 at full resolution.

The encoded feature representations may subsequently be provided to a decoder pathway via the plurality of indicated skip connections 517 and the bridge layer 514.

The bridge layer 514 may be configured to facilitate interactions between the other layers and blocks. The bridge layer 514 is a component of the regression model that connects the transformer encoder pathway to the CNN decoder pathway at the lowest resolution level of the U-Net like structure. In some embodiments, the bridge layer 514 receives the most deeply encoded feature representations from the final stage of the plurality of transformer blocks 512 after processing through the plurality of patch merging layers 513.

The bridge layer 514 may serve as the bottleneck of the encoder-decoder architecture, where the encoded features from the input MRI image 501 are at their most compressed spatial resolution with the highest channel dimensionality. The bridge layer 514 transforms these encoded features into a format suitable for the decoder pathway, facilitating the transition from the transformer-based encoder to the CNN-based decoder.

The bridge layer 514 works in conjunction with the plurality of indicated skip connections 517, which transfer feature information from the encoder stages directly to corresponding decoder stages at matching resolutions. In some embodiments, the bridge layer 514 provides deep semantic features that capture global contextual information. These deep features may be progressively upsampled and combined with the plurality of residual block layers 515 and the hidden layer(s) 516 to reconstruct the electric field map 503 at full resolution.

The plurality of residual block layers 515 may be components of the CNN decoder within the regression model that progressively reconstruct the electric field map 503 from the encoded feature representations. The plurality of residual block layers 515 operate within the decoder pathway of the U-Net like structure, where a transformer encoder and CNN decoder work together to perform image-to-image translation from the input MRI image 501 to the electric field map 503.

The plurality of residual block layers 515 may be configured to receive a plurality of inputs, potentially via a plurality of channels. In at least some embodiments, the plurality of residual block layers 515 are configured to compress the inputs with a 1×1 convolution such that input and output can be added.

Each of the plurality of residual block layers 515 may employ residual connections that add the input of the block to its output, enabling more effective gradient flow during training and allowing the network to learn residual mappings rather than direct transformations. The plurality of residual block layers 515 apply convolutional operations to progressively upsample and refine the feature representations at each resolution level of the decoder pathway.

The plurality of residual block layers 515 may receive features from the bridge layer 514 and from the plurality of indicated skip connections 517, which transfer encoder features from corresponding resolution levels of the plurality of transformer blocks 512. By combining the deeply encoded semantic features from the bridge layer 514 with the fine-grained spatial details preserved through the plurality of indicated skip connections 517, the plurality of residual block layers 515 progressively reconstruct the spatial resolution while refining the predicted electrical current distribution. The output of the plurality of residual block layers 515 is provided to the hidden layer(s) 516 for final processing to generate the electric field map 503.

The hidden layer(s) 516 may each be components of the CNN decoder within the regression model that perform final processing to generate the electric field map 503 from the reconstructed feature representations. The hidden layer(s) 516 may be positioned at the output stage of the decoder pathway, receiving features from the plurality of residual block layers 515 after progressive upsampling through the U-Net like structure.

In some embodiments, the hidden layer(s) 516 transform the multi-channel feature representations into the final output format of the electric field map 503, which predicts the electrical current dosages that reach the target treatment areas. The hidden layer(s) 516 map the learned feature representations to voxel-wise electric field intensity values, completing the image-to-image translation from the three-dimensional (3D) input MRI image 501 to the electric field map 503.

The hidden layer(s) 516 may work in conjunction with the plurality of residual block layers 515 and receive information that has been progressively refined through the decoder pathway. The features provided to the hidden layer(s) 516 incorporate both the global contextual information from the bridge layer 514 and the fine-grained spatial details preserved through the plurality of indicated skip connections 517 from the plurality of transformer blocks 512. The downstream hidden layer(s) 516 may produce the final electric field map 503 output that captures both the brain structure and current doses for the specified electrode montage.

The hidden layer(s) 516 may be configured to learn hierarchical representations of data within the input MRI image 501 and its various intermediate forms as provided by the other layers of the example method pipeline segment 500. In at least some embodiments, each of the hidden layer(s) 516 is configured for a feature extraction iteration. Each of the hidden layer(s) 516 may be configured to receive input from a previous layer, apply a weighted sum and bias, and pass the result through an activation function to produce an output. In at least some embodiments, the weights and biases are learned during the training process, allowing the network to adjust its behavior to better predict a desired output.

As depicted, example method pipeline segment 500 yields logits 502, comprising raw, unnormalized output values from the hidden layer(s) 516. The logits 502 are the output of the classification model that represent the unnormalized prediction scores for classifying the electric field map 503 into the different electrode montages. The classification model classifies the regression outputs into the different electrode montages, and the logits 502 represent the raw output values prior to conversion into probability distributions.

The logits 502 may provide an additional regularization for the regression output by enabling the classification model to classify the output maps according to electrode montage type. This regularization helps further differentiate the electric field maps that result from the same input MRI image 501 when different electrode montage configurations are applied.

In some embodiments, the logits 502 are used to compute the cross entropy loss component of the loss function, where the output electrode montage class is compared against the true electrode montage class. The entire loss function is backpropagated across both the regression and classification networks, enabling the dual learning scheme where the regression and classification models train simultaneously.

In some embodiments, the logits 502 are combined with a one-hot montage vector 505. The one-hot montage vector 505 may be an input representation that encodes a user's choice for the electrode montage that is used to produce the electric field maps. The one-hot montage vector 505 may comprise a sparse binary vector where a single element is set to one to indicate the selected electrode montage class, while all other elements are set to zero.

The one-hot montage vector 505 enables users to enter a desired electrode montage in addition to the input MRI image 501 for a custom output. The system supports multiple electrode montage configurations, such as F3-F4 and C3-Fp2 montages, which are common configurations used in tDCS research. Each distinct electrode montage configuration may correspond to a unique position in the one-hot montage vector 505.

The one-hot montage vector 505 may be provided to a head block 504, which conditions the regression model to generate the electric field map 503 specific to the selected electrode montage. By incorporating the one-hot montage vector 505, the system allows users to input both a unique MRI and a desired electrode montage to produce tailored electric field predictions. This enables the pipeline to help clinical research trials determine optimal individual tDCS parameters by differentiating the electric field maps that result from the same input MRI image 501 based on different stimulation configurations.

In at least some embodiments, the head block 504 is configured to process the combination of the logits 502 and the resized one-hot montage vector 505 using (i) a 3×3×3 convolution, (ii) a batch normalization mechanism, and (iii) a rectified linear unit (ReLU) function. The head block 504 may be a component of the regression model that receives and processes a vector corresponding to a user's choice for the electrode montage that is used to produce the electric field maps. The head block 504 enables the system to allow users to input both a unique MRI and a desired electrode montage to produce tailored electric field predictions.

The head block 504 may condition the regression model to generate electric field maps specific to the selected electrode montage configuration, such as F3-F4 or C3-Fp2 montages. By incorporating the electrode montage vector, the head block 504 enables the regression model to differentiate the electric field maps that result from the same input MRI image 501 based on different stimulation configurations.

The head block 504 may work in conjunction with the encoder-decoder architecture of the regression model, providing electrode montage context that influences the generation of the electric field map 503. This conditioning mechanism enables the pipeline to help clinical research trials determine optimal individual tDCS parameters by allowing users to enter a desired electrode montage in addition to the unique MRI for a custom output.

FIG. 5B provides an example method pipeline segment 520 in accordance with some embodiments of the present disclosure. As depicted, example method pipeline segment 520 includes a plurality of MBConv1 blocks 521 and a plurality of MBConv6 blocks 522. Generally, each of the MBConv1 blocks 521 and the MBConv6 blocks 522 are configured for (i) expansion convolution, (ii) depthwise convolution, and (iii) projection convolution.

The plurality of MBConv1 blocks 521 may be mobile inverted bottleneck convolutional blocks within an EfficientNet framework of the classification model that employ a 3×3 kernel size for depthwise separable convolutions. The plurality of MBConv1 blocks 521 may be part of the method pipeline segment 520 and may integrate squeeze-and-excitation blocks, depthwise separable convolutions, and inverted residuals.

The plurality of MBConv1 blocks 521 may contribute to the classification model's ability to classify the electric field map 503 into the different electrode montages. The mobile inverted bottleneck architecture may first expand the input channels through a pointwise convolution, apply the 3×3 depthwise separable convolution to extract spatial features, and then project back to a lower-dimensional output through another pointwise convolution. The squeeze-and-excitation blocks within the plurality of MBConv1 blocks 521 may adaptively recalibrate channel-wise feature responses by modeling interdependencies between channels.

The plurality of MBConv1 blocks 521 may work in conjunction with the plurality of MBConv6 blocks 522 within the EfficientNet framework, which may be designed to improve accuracy while conserving computational resources through a special model scaling scheme that balances network depth, width, and resolution. The classification model may serve as an additional regularization for the regression output, classifying the output maps according to electrode montage type to generate the logits 502.

The plurality of MBConv6 blocks 522 may be mobile inverted bottleneck convolutional blocks within the EfficientNet framework 532 of the classification model that employ a 5×5 kernel size for depthwise separable convolutions. The plurality of MBConv6 blocks 522 may be part of the method pipeline segment 520 and may integrate squeeze-and-excitation blocks, depthwise separable convolutions, and inverted residuals.

The plurality of MBConv6 blocks 522 may utilize a larger expansion ratio compared to the plurality of MBConv1 blocks 521, expanding the input channels by a factor of six before applying the depthwise separable convolution. The 5×5 kernel size may enable the plurality of MBConv6 blocks 522 to capture larger spatial patterns within the electric field map 503 during classification. The squeeze-and-excitation blocks within the plurality of MBConv6 blocks 522 may adaptively recalibrate channel-wise feature responses by modeling interdependencies between channels.

The plurality of MBConv6 blocks 522 may work in conjunction with the plurality of MBConv1 blocks 521 within the EfficientNet framework, which may be designed to improve accuracy while conserving computational resources through a special model scaling scheme that balances network depth, width, and resolution.

In some embodiments, a subject block expands an input's channel dimension using a 1×1 convolution in the case of an MBConv1 block 521 or a 6×1 convolution in the case of an MBConv6 block 522. This expansion phase allows the input to be transformed into a higher-dimensional space, enabling the extraction of more complex features. A subject block is then configured to apply a 3×3 depthwise convolution operating on each input channel separately, thereby extracting spatial features from the input at a minimized computational cost. Further, the subject block is then configured to reduce the dimensionality of the feature map using a 1×1 project convolution, thereby ensuring that the output of the subject block is a manageable size. As depicted, the combination of MBConv1 blocks 521 and MBConv6 blocks 522 ultimately processes the electric field map 503 into an electrode montage class.

FIG. 5C provides an example method pipeline segment 530. As depicted, the example method pipeline segment 530 depicts processing of an input MRI image 501 using a Swin UNETR framework 531 to provide an electric field map 503. The Swin UNETR framework 531 may be a regression model architecture within the method pipeline segment 530 that performs image-to-image translation from the input MRI image 501 to the electric field map 503. The Swin UNETR framework 531 may be based on the principle of combining the advantages of U-Net with those of transformer modules in a U-Net like structure.

The Swin UNETR framework 531 may employ a transformer encoder and CNN decoder together, where the transformer encoder includes the patch partition layer 511, the plurality of transformer blocks 512, and the plurality of patch merging layers 513. The Swin UNETR framework 531 may improve upon prior architectures by adding encoder feature extraction at five different resolutions using shifting windows, enabling the plurality of transformer blocks 512 to learn global spatial information through attention mechanisms.

The Swin UNETR framework 531 may compensate for the limited ability of convolutional neural networks (CNNs) to model global information due to the limited size of convolution kernels. The decoder pathway of the Swin UNETR framework 531 may include the bridge layer 514, the plurality of residual block layers 515, and the hidden layer(s) 516, which progressively reconstruct the electric field map 503. The plurality of indicated skip connections 517 may transfer feature information from the encoder stages to corresponding decoder stages at matching resolutions.

The Swin UNETR framework 531 may be modified to receive the one-hot montage vector 505 via the head block 504, enabling the regression model to generate electric field maps specific to a user's choice for the electrode montage.

In at least some embodiments, the Swin UNETR framework additionally determines a mean absolute error, and an expected calibration error associated with the electric field map. Further, the example method pipeline segment 530 depicts processing an electric field map using an EfficientNet framework 532 to provide an electrode montage class and a corresponding cross entropy, wherein the cross entropy measures an average number of bits needed to identify an item drawn from a set when the coding scheme used for the set is optimized for an estimated probability distribution rather than a true distribution. The EfficientNet framework 532 may be the classification model architecture within the method pipeline segment 520 that classifies the electric field map 503 into the different electrode montages. The EfficientNet framework 532 may serve as an additional regularization for the regression output, classifying the output maps according to electrode montage type to help further differentiate the electric field maps that result from the same input MRI image 501.

The EfficientNet framework 532 may be designed to improve accuracy while conserving computational resources through a special model scaling scheme that balances network depth, width, and resolution. The EfficientNet framework 532 may include the plurality of MBConv1 blocks 521 and the plurality of MBConv6 blocks 522, which employ mobile inverted bottleneck convolutional blocks that integrate squeeze-and-excitation blocks, depthwise separable convolutions, and inverted residuals.

The EfficientNet framework 532 may receive the electric field map 503 generated by the Swin UNETR framework 531 and process it through the method pipeline segment 520 to generate the logits 502. The logits 502 may represent the output electrode montage class predictions, which may be compared against the true electrode montage class during training using cross entropy loss. The entire loss function may be backpropagated across both the Swin UNETR framework 531 and the EfficientNet framework 532, enabling a dual learning scheme where the regression and classification models train simultaneously. As depicted, the example method pipeline segment 530 additionally includes backpropagating all losses with respect to both the Swin UNETR framework 531 and the EfficientNet framework 532.

### IV. EXAMPLES OF CERTAIN TERMS

In some embodiments, the terms “user system,” “user device,” “user computing device,” “user computing entity,” and the like may refer to any system configured to run an EBS optimization method as described herein. In general, the user system may refer to any system capable of providing an interface to a user device via which a user can interact with an EBS optimization application. User systems as described herein enable a user to view electrode performance information, view data received from an EBS device, and/or interact with one or more selectable features corresponding to an EBS framework.

In some embodiments, the term “subject” refers to an individual whose head and/or brain are being scanned or otherwise imaged for analysis. In at least some embodiments, the term subject is used to refer to an individually actively connected to/exposed to a precision dosing system configured to administer electrical stimulation of the brain.

In some embodiments, the term “electrical-stimulation device,” as used in relation to the brain herein, refers to a tool that delivers electrical signals to specific brain regions or nerves to modulate brain activity or treat certain conditions. Example electrical-stimulation devices are configured to administer transcranial stimulation (TMS), deep brain stimulation (DBS), transcranial electrical stimulation (tES), vagus nerve stimulation (VNS), cranial electrotherapy stimulation (CES), and the like.

In some embodiments, the term “classification model” refer to a machine learning model configured to categorize data points into predefined groups or classes based on learned patterns for input data. Classification models as used herein refer to models which aim to predict which class (such as an electron montage class, for example) a new, unseen data point belongs to based on its features. Types of classification models include binary classification models, multiclass classification models, and multilabel classification models.

In some embodiments, the term “regression model” is used to refer to a supervised learning technique used to predict a continuous numerical value (the dependent variable) based on one or more independent features (the independent variables). Regression models learn the relationships between these variables according to historical data to make predictions. Ultimately, regression models/algorithms attempt to plot a best-fit line or curve amongst one or more data.

In some embodiments, the term “electric field map,” as it relates to the brain, refers to a visual representation or technique that maps and analyzes electrical activity within the brain. In at least some embodiments, the electric field map of the brain is configured to visualize brain function and identify areas of activity or dysfunction.

In some embodiments, the term “electrode montage,” as it relates to EBS, refers to the specific arrangement and placement of electrodes on the scalp or body to record or stimulate electrical activity. An electrode montage may additionally comprise a specific method of connecting electrodes to recording channels of an electroencephalograph (EEG) or other neurostimulation device to determine how electrical signals are arranged and displayed. Examples of electrode montages include the 10-20 system, which is a standardized system for electrode placement widely used in EEG recordings. Additional example electrode montages include bipolar and referential montages. Electrode montages may specifically define anode and cathode placement to induce specific effects on brain activity.

In some embodiments, the term “digital twin,” as it relates to EBS, refers to a generated brain mapping configured to mirror an actual brain for use with a simulator configured to simulate the effects of various EBS operations on the actual brain. In general, digital twins as used herein are virtual models of a subject brain configured to help predict how the brain would react to different treatments. A digital twin may be continuously updated with real-time data, reflecting the current state of the physical entity (the brain).

### V. EXAMPLE SYSTEM OPERATIONS

FIG. 6 depicts an example process 600 for generating electrical current maps in accordance with at least some example embodiments of the present disclosure. As depicted, example process 600 includes receiving (602) one or more neuroimaging datasets, generating (604) reference electric field maps associated with the neuroimaging datasets, preprocessing (606) one or more subject images, generating (608) electric field maps using a regression model, classifying (610) the electric field maps into one or more electrode montages, and configuring (612) an EBS operation according to the electric field maps. Example process 600 may enable generation of electric field maps based on optimized EBS operation based on one or more neuroimaging datasets.

At step 602, system 150 receives one or more neuroimaging datasets. In at least some embodiments, receiving (602) one or more neuroimaging datasets includes receiving data indicating EBS effects on cognition. In at least some embodiments, receiving (602) one or more neuroimaging datasets includes receiving one or more processed brain images and a corresponding set of cognitive results for the one or more processed brain images. Receiving (602) one or more neuroimaging datasets may include receiving a first dataset corresponding to brain images of older adults (i.e., older adults between 65-89 years old, for example) and a second dataset corresponding to brain images of younger adults (i.e., adults between 22-35). In at least some embodiments, the received one or more neuroimaging dataset may include behavioral and 3T MRI data corresponding to each subject.

At step 604, system 150 generates reference electric field maps associated with the neuroimaging datasets. In at least some embodiments, generating (604) reference electric field maps associated with the neuroimaging datasets includes inputting the neuroimaging datasets to an EBS simulation system or application. An appropriate EBS simulation application may be configured to (i) simulate the electric fields generated during EBS, (ii) take MRI images as input and use them to create a realistic model of the head, including different tissue types, and/or (iii) automatically segment the head into different tissues, place virtual electrodes for EBS, generate a finite element mesh, and determine voltage and electric field distributions. One example of an appropriate EBS simulation application is Realistic vOlumetric-Approach-based Simulator for Transcranial electric stimulation (or “ROAST”). In at least some embodiments, generating reference electric field maps includes selecting one or more electrode montages for simulating the EBS operation(s). An electrode montage as used herein refers to the specific arrangement and pairing of electrodes to display and analyze brain electrical activity, thereby enabling visualization and interpretation of electroencephalography data. In at least some embodiments, common montages such as F3-F4 (wherein “F” indicates the frontal lobe, “3” indicates a specific location along the midline that 70% of the distance from the nasion to the vertex and 25% from the midline to the right, and “4” indicates a location on the right side of the brain, specifically over the right dorsolateral prefrontal cortex) and/or C3-Fp2 (wherein “C3” indicates a central region of the scalp approximately 20% of the distance between the nasion between the eyebrows and the inion at the base of the skull, and 20% of the distance from the midline position “Cz” towards the left ear, and “Fp2” indicates the frontal pole 2, located on the right frontal pole just above the eyebrows). In at least some embodiments, the EBS simulation application is configured according to a standard input current amplitude (such as 2 mA). Although the example embodiments above discussed specific values for various parameters of the EBS (e.g. current intensity value, number of stimulation sessions, period time over which the sessions were conducted, conductivity values of the electrode and gel layers, etc.) these parameter values are not limiting and in other example embodiments, one or more of these parameter values can be adjusted or varied.

At step 606, system 150 preprocesses one or more subject images. Preprocessing (606) one or more subject images may include receiving one or more subject images for analysis using system 150. In at least some embodiments, the received set of subject images includes inference MRI images. Preprocessing (606) one or more subject images may include utilizing at least one Medical Open Network for Artificial Intelligence (MONAI) transformation to provide a preprocessed set of subject images. MONAI is an open-source framework to help optimize deep learning for healthcare imaging that comes with optimized frameworks for image preprocessing, deep learning, and evaluation. In at least some embodiments, preprocessing (606) one or more subject images includes normalizing all MRI voxels between 0-1, crops images into 128×128×128 inputs, and performs data augmentation on one or more training data (such as the received neuroimaging datasets). In at least some embodiments, the training data has random data flips and intensity shifts both at a probability of 0.1. In at least some embodiments, evaluation and testing are evaluated over the entire image inputs using 128×128×128 windows using a sliding window function.

At step 608, system 150 generates electric field maps corresponding to the one or more subject images using a regression model. Generating (608) electric field maps using a regression model may include performing image-to-image translation from an image of the one or more subject images to a 3D electric field map output. In at least some embodiments, system 150 is configured to input a vector that corresponds to a user's choice for an electrode montage that is used to produce the electric field maps. In at least some embodiments, generating (608) electric field maps using a regression model includes leveraging a combination of U-Net architectures and transformer modules. U-Net architectures are convolutional neural networks developed for image segmentation. U-Net architectures supplement a usual contracting network by successive layers, where pooling operations are replaced by unsampling operators. The U-Net architectures consist of a contracting path and an expansive path, wherein the contracting path is a typical convolutional network that consists of repeated application of convolutions, each followed by a rectified linear unit (ReLU) and a max pooling operation. During the contraction, the spatial information is reduced while feature information is increased. The expansive pathway combines the feature and special information through a sequence of up-convolutions and concatenations with high-resolution features from the contracting path. In at least some embodiments, SwinUNETR is employed as the basic regression model architecture. In general, the regression model is configured to employ a transformer encoder and a convolutional neural network together in a U-Net like structure. In some embodiments, an encoder feature extraction is added at different resolutions using shifting windows.

At step 610, system 150 classifies the electric field maps into one or more electrode montages. Classifying (610) the electric field maps into one or more electrode montages may include inputting the electric field maps into a classification model. Classifying (610) the electric field maps into one or more electrode montages regularizes the regression model to help further differentiate electric field maps that result from a same MRI. In at least some embodiments, a convolutional neural network architecture and scaling method configured to uniformly scale all dimensions of depth/width/resolution using a compound coefficient is utilized for classification. EfficientNet is one example of an appropriate classification algorithm for use with the techniques as described. In at least some embodiments, the classification model is configured to use a mobile inverted bottleneck convolutional block that integrates squeeze-and-excitation blocks, depthwise separable convolutions, and inverted residuals to classify the regression outputs into one or more electrode montages.

At step 612, system 150 configures an EBS operation according to the electric field maps. Configuring (612) an EBS operation according to the electric field maps may include identifying one or more optimal EBS operations based at least in part on the classified electric field maps. In at least some embodiments, configuring (612) an EBS operation according to the electric field maps includes causing execution of an EBS operation. In at least some embodiments, configuring (612) an EBS operation according to the electric field maps includes configuring one or more parameters of an EBS operation according to the classified electric field maps.

In at least some embodiments, the regression model and/or the classification model may be adjusted according to a custom loss function. The loss function contains three components to balance electric field map accuracy, sensitivity to electrode montage type, and calibration. The loss function is defined according to equation 1 below:

L=α×MAE(y, \hat{ }y)+β×CrossEntropy(v, \hat{ }v)+γ×ECE(y, \hat{ }y) (1)

wherein y is the output electric field map, \hat{ }y is the reference electric field map, v is the output electrode montage class, and \hat{ }v is the true electrode montage class, and MAE is the mean Absolute Error. ECE is the expected calibration error determined according to equation 2 below, which is a modified ECE equation configured to handle regression problems. The modified equation takes the residual differences between the predicted and target values, then it bins them according to the ranges of the predicted values. α, β, and γ are weighting terms determined using a simple grid search to establish the best weightings for each loss. In at least some embodiments, the optimal weightings are α=1.0, β=0.5 and γ=0.5. According to equation 2, confidence values are binned into M bins in which Binᵢ represents a current bin, aᵢ represents current accuracy, and cᵢ represents the current confidence. For regression, the prediction values are binned, and the residual error is used as a measure of accuracy.

In at least some embodiments, process 600 additionally includes configuring a digital twin according to the electric field maps. System 150 may be configured to generate a virtual model of the subject brain according to the electric field maps. In at least some embodiments, the virtual model is subjected to one or more EBS operations in accordance with the electric field maps to predict how the subject brain will react to the one or more EBS operations.

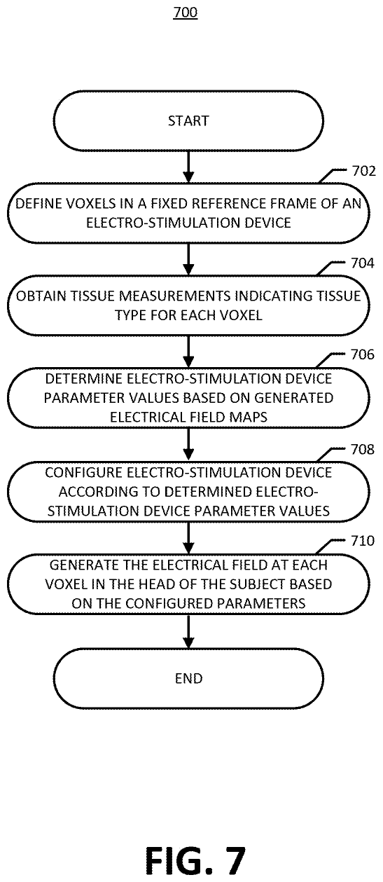

FIG. 7 is a flowchart diagram of an example process 700 for determining parameter values of the electro-stimulation device 170 on a subject 190 to achieve improved cognitive function, in accordance with at least some example embodiments of the present invention. Example process 700 method determines one or more parameter values (e.g. current intensity, electrode placement, etc.) of the electro-stimulation device 170 for a new subject to improve a cognitive response of the new subject.

In an embodiment, in steps 702 and 704, system 150 defines the voxels of a subject image and obtains tissue measurements to indicate the tissue type of each voxel within the subject 110. In step 702, the one or more voxels within the frame of reference of the electro-stimulation device 170 are determined. In step 704, tissue measurements are obtained that indicate the tissue type of each voxel in the one or more voxels in the head 160 of the subject 110. In an example embodiment, steps 202 and 204 are performed using individual Tl-weighted images that are converted from Digital Imaging and Communications in Medicine (DICOM) format to Neuroimaging Informatics Technology Initiative (NIfTI) format. In an example embodiment, the computational models of current density are computed using the ROAST toolbox with parallel processing on a high-performance cluster. In one embodiment, segmentation is carried out to classify tissue types into gray and white matter. Segmentations may be visually inspected and manually corrected for errors before reprocessing. Segmentations may be combined with segmented CSF, bone, skin, and air from ROAST. In one embodiment, segmentation quality may be visually inspected by overlaying segmented masks onto their respective Tl-weighted images.

In an embodiment, in step 706 the value of the parameters of the electric field determined according to example process 600, for example, are obtained (e.g. retrieved from stored memory of the system 150). In an embodiment, in step 708, a value of one or more parameters of the electro-stimulation device 170 is determined based on the parameter values of the electric field and the tissue measurements of the new subject. In an embodiment, in step 708, after determining the one or more parameter values of the electro-stimulation device 170, the system 150 configures the electro-stimulation device 170 based on these parameter values. In an example embodiment, one or more input devices of the system 150 are used to input the current intensity value such that the system 150 transmits a signal to the electro-stimulation device 170 with that current intensity. In other embodiments, in the cathode or anode are displaced (e.g. relative to an ideal F3/F4 position). In an embodiment, in step 710 the electric field is generated by the electro-stimulation device 170 at each voxel of the subject.

### VI. CONCLUSION

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

### VII. EXAMPLES

Example 1. A computer-implemented method comprising: receiving one or more neuroimaging datasets; generating one or more reference electric field maps associated with the one or more neuroimaging datasets; preprocessing one or more subject images depicting a subject brain; generating, using a regression model, one or more electric field maps corresponding to the one or more subject images; classifying, using a classification model, the one or more electric field maps into one or more electrode montages; and determining one or more electrical brain stimulation parameters corresponding to the one or more electrode montages.

Example 2. The computer-implemented method of Example 1, wherein the regression model is a SwinUNETR model architecture.

Example 3. The computer-implemented method of any of the preceding Examples, wherein the classification model is an EfficientNet classification model.

Example 4. The computer-implemented method of any of the preceding Examples, wherein the one or more reference electric field maps are generated using a ROAST.

Example 5. The computer-implemented method of any of the preceding Examples, wherein the one or more electrical brain stimulation parameters correspond to one or more transcranial electrical stimulation operations.

Example 6. The computer-implemented method of any of the preceding examples, wherein the one or more electrical brain stimulation parameters correspond to one or more transcranial direct current stimulation operations.

Example 7. The computer-implemented method of any of the preceding examples, further comprising transmitting the one or more electrical brain stimulation parameters to an electro-stimulation device, wherein the electro-stimulation device is one of a transcranial magnetic stimulation device, an electroconvulsive therapy device, a deep brain stimulation device, a transcranial alternative current device, a transcranial random noise stimulation device.

Example 8. A computing apparatus comprising memory and one or more processors communicatively coupled to the memory, the one or more processors configured to perform operations comprising: receiving one or more neuroimaging datasets; generating one or more reference electric field maps associated with the one or more neuroimaging datasets; preprocessing one or more subject images depicting a subject brain; generating, using a regression model, one or more electric field maps corresponding to the one or more subject images; classifying, using a classification model, the one or more electric field maps into one or more electrode montages; and determining one or more electrical brain stimulation parameters corresponding to the one or more electrode montages.

Example 9. The computing apparatus of example 8, wherein the regression model is a SwinUNETR model architecture.

Example 10. The computing apparatus of any of the preceding examples, wherein the classification model is an EfficientNet classification model.

Example 11. The computing apparatus of any of the preceding examples, wherein the one or more reference electric field maps are generated using a ROAST.

Example 12. The computing apparatus of any of the preceding examples, wherein the one or more electrical brain stimulation parameters correspond to one or more transcranial electrical stimulation operations.

Example 13. The computing apparatus of any of the preceding examples, wherein the one or more electrical brain stimulation parameters correspond to one or more transcranial direct current stimulation operations.

Example 14. The computing apparatus of any of the preceding examples, wherein the one or more subject images are MRIs.

Example 15. One or more non-transitory computer-readable storage media including instructions that, when executed by one or more processors, cause the one or more processors to perform operations comprising: receiving one or more neuroimaging datasets; generating one or more reference electric field maps associated with the one or more neuroimaging datasets; preprocessing one or more subject images depicting a subject brain; generating, using a regression model, one or more electric field maps corresponding to the one or more subject images; classifying, using a classification model, the one or more electric field maps into one or more electrode montages; and determining one or more electrical brain stimulation parameters corresponding to the one or more electrode montages.

Example 16. The one or more non-transitory computer-readable storage media of example 15, wherein the regression model is a SwinUNETR model architecture.

Example 17. The one or more non-transitory computer-readable storage media of any of the preceding examples, wherein the classification model is an EfficientNet classification model.

Example 18. The one or more non-transitory computer-readable storage media of any of the preceding examples, wherein the one or more reference electric field maps are generated using a ROAST.

Example 19. The one or more non-transitory computer-readable storage media of any of the preceding examples, wherein the one or more electrical brain stimulation parameters correspond to one or more transcranial electrical stimulation operations.

Example 20. The one or more non-transitory computer-readable storage media of any of the preceding examples, wherein the one or more neuroimaging datasets are received from an imaging device, and wherein the imaging device is one of an X-ray Computed tomography CT scanner, a nuclear MRI scanner or a 4DCT based imaging system.

1. A computer-implemented method comprising:
generating, by one or more processors, one or more reference electric field maps associated with one or more neuroimaging datasets;
preprocessing, by the one or more processors, one or more subject images;
generating, by the one or more processors and using a regression model, one or more electric field maps corresponding to the one or more subject images;
classifying, by the one or more processors and using a classification model, the one or more electric field maps into one or more electrode montages; and
determining, by the one or more processors, one or more electrical brain stimulation parameters corresponding to the one or more electrode montages.

2. The computer-implemented method of claim 1, wherein the regression model is a SwinUNETR model architecture used to predict the one or more electric field maps.

3. The computer-implemented method of claim 1, wherein the classification model is an EfficientNet classification model used to predict the one or more electrode montages.

4. The computer-implemented method of claim 1, wherein the one or more reference electric field maps are generated using a Realistic vOlumetric, Approach-based Simulator for Transcranial electric stimulation (ROAST).

5. The computer-implemented method of claim 1, wherein the one or more electrical brain stimulation parameters correspond to one or more transcranial electrical stimulation operations.

6. The computer-implemented method of claim 1, wherein the one or more electrical brain stimulation parameters correspond to one or more transcranial direct current stimulation operations.

7. The computer-implemented method of claim 1, further comprising transmitting the one or more electrical brain stimulation parameters to an electro-stimulation device, wherein the electro-stimulation device is one of a transcranial magnetic stimulation device, an electroconvulsive therapy device, a deep brain stimulation device, a transcranial alternative current device, a transcranial random noise stimulation device.

8. A computing apparatus comprising memory and one or more processors communicatively coupled to the memory, the one or more processors configured to perform operations comprising:
generating one or more reference electric field maps associated with one or more neuroimaging datasets;
preprocessing one or more subject images depicting a subject brain;
generating, using a regression model, one or more electric field maps corresponding to the one or more subject images;
classifying, using a classification model, the one or more electric field maps into one or more electrode montages; and
determining one or more electrical brain stimulation parameters corresponding to the one or more electrode montages.

9. The computing apparatus of claim 8, wherein the regression model is a SwinUNETR model architecture used to predict the one or more electric field maps.

10. The computing apparatus of claim 8, wherein the classification model is an EfficientNet classification model used to predict the one or more electrode montages.

11. The computing apparatus of claim 8, wherein the one or more reference electric field maps are generated using a Realistic vOlumetric, Approach-based Simulator for Transcranial electric stimulation (ROAST).

12. The computing apparatus of claim 8, wherein the one or more electrical brain stimulation parameters correspond to one or more transcranial electrical stimulation operations.

13. The computing apparatus of claim 8, wherein the one or more electrical brain stimulation parameters correspond to one or more transcranial direct current stimulation operations.

14. The computing apparatus of claim 8, wherein the one or more subject images are MRIs.

15. One or more non-transitory computer-readable storage media including instructions that, when executed by one or more processors, cause the one or more processors to perform operations comprising:
generating one or more reference electric field maps associated with one or more neuroimaging datasets;
preprocessing one or more subject images depicting a subject brain;
generating, using a regression model, one or more electric field maps corresponding to the one or more subject images;
classifying, using a classification model, the one or more electric field maps into one or more electrode montages; and
determining one or more electrical brain stimulation parameters corresponding to the one or more electrode montages.

16. The one or more non-transitory computer-readable storage media of claim 15, wherein the regression model is a SwinUNETR model architecture used to predict the one or more electric field maps.

17. The one or more non-transitory computer-readable storage media of claim 15, wherein the classification model is an EfficientNet classification model used to predict the one or more electrode montages.

18. The one or more non-transitory computer-readable storage media of claim 15, wherein the one or more reference electric field maps are generated using a Realistic vOlumetric, Approach-based Simulator for Transcranial electric stimulation (ROAST).

19. The one or more non-transitory computer-readable storage media of claim 15, wherein the one or more electrical brain stimulation parameters correspond to one or more transcranial electrical stimulation operations.

20. The one or more non-transitory computer-readable storage media of claim 15, wherein the one or more neuroimaging datasets are received from an imaging device, and wherein the imaging device is one of an X-ray Computed tomography (CT) scanner, a nuclear MRI scanner or a four-dimensional computed tomography (4DCT) based imaging system.
